(19) United States
(12) Patent Application Publication
(10) Pub. No.: US 2026/0116902 A1
(43) Pub. Date: Apr. 30, 2026

(54) NOVEL COMPOUND AS FUNCTIONAL ANTAGONIST FOR S1PR1 AND S1PR4

(71) Applicant: NEXTGEN BIOSCIENCE CO., LTD., Seongnam-si (KR)

(72) Inventors: Bongyong LEE, Seoul (KR); Yanghae Park, Seoul (KR); Eunjeong Kim, Seongnam-si (KR); Hojung Choi, Incheon (KR)

(73) Assignee: NEXTGEN BIOSCIENCE CO., LTD., Seongnam-si (KR)

(21) Appl. No.: 18/933,189

(22) Filed: Oct. 31, 2024

(51) Int. Cl. C07F 9/6518; A61K 31/675; A61P 1/00

(52) U.S. Cl. CPC C07F 9/6518; A61K 31/675; A61P 1/00

(57) ABSTRACT

The present invention relates to a novel compound acting as a functional antagonist for S1PR1 and S1PR4, and a use thereof. The compound according to the present invention exhibits significantly excellent binding strength to S1PR1 and S1PR4 receptors, thus exhibiting an excellent effect in preventing or treating inflammatory bowel disease, alopecia areata (AA), or an autoimmune disease.

### BACKGROUND

### 1. Field of the Invention

The present invention relates to a novel compound that acts as a functional antagonist for S1PR1 and S1PR4, and a use thereof.

### 2. Discussion of Related Art

An S1PR1 receptor is mainly present in lymphocytes and plays a role in regulating the release of lymphocytes from immune cells. An S1PR4 receptor is also present in lymphocytes so it plays a role in not only immune cell trafficking but also in T cell regulation, and thus is involved in inflammatory responses.

Among anti-inflammatory diseases, multiple sclerosis (MS) is the most common chronic inflammatory demyelinating disease in the central nervous system (the brain, the spinal cord, and the optic nerve). It is pathologically characterized by multiple inflammatory and demyelination mainly in the white matter of the central nervous system. Although the cause of the disease is not yet clear, it is considered to be an autoimmune disease caused by the surrounding environment in genetically susceptible patients.

Multiple sclerosis (MS) treatment may be largely divided into acute treatment represented by high-dose steroids, long-term disease modifying therapy, and symptomatic therapy. In MS treatment, above all, long-term disease relief that reduces repeated relapse and impairment is important. Conventionally, although interferon has been used as a therapeutic for MS, MS is not completely cured due to repetitive relapse and remission, leaving disability and various neurological symptoms depending on the area where the demyelinating lesion occurs. MS patients with no specific neurological impairment and those progressing to severe disability exhibit very different symptoms and disease progression.

Thus, for over 100 years, there has been no clear treatment to reduce the repeated relapses of MS. However, recently, fingolimod (FTY720, Gilenya), which exhibits a therapeutic effect by acting on the sphingosine-1-phosphate (S1P) receptor of T-type white blood cells and effectively blocking the migration to the inflammatory site, has started to be used as the first oral therapeutic for MS.

Specifically, FTY720 is an oral immunoregulatory drug that is taken once a day and a synthetic analog of sphingosine-1-phosphate (S1P). It is an MS therapeutic with a mechanism of action in which some of the lymphocytes in the lymph nodes are reversibly captured to sequester the lymphocyte in a secondary lymphoid organ and to inhibit entry into the central nervous system or reduce the number of lymphocytes circulating in the blood stream, resulting in a reduction in the number of activated lymphocytes and reducing inflammatory destruction.

Here, FTY720, as a sphingosine 1-phosphate (S1P) receptor modulator, is a compound acting non-selectively on one or more S1P receptors, including S1PR1, S1PR2, S1PR3, S1PR4, and S1PR5. When FTY720 binds to such an S1P receptor, an intracellular heterotrimeric G-protein dissociates into Gα-GTP and Gβγ-GTP, which can regulate downstream signaling pathways and kinases, which is useful for the treatment of diseases that are mediated by lymphocyte interactions, such as transplant rejection, autoimmune diseases, infectious diseases, or cancer, diseases or disorders mediated by lymphocyte interactions, for example, autoimmune diseases, particularly, Hashimoto's thyroiditis, or peripheral neuropathies, such as Guillain-Barré syndrome (GBS), multifocal motor neuropathy (MMN) with conduction block, or paraproteinemic demyelinating peripheral neuropathy (PDN), or treatment or prevention of MS.

Like this, FTY720, approved as an oral medication for MS, is attracting attention for its mechanism of action that modulates immunity by simply regulating lymphocytes without depletion caused by the cell death of lymphocytes, but has problems due to severe side effects centered on the cardiac circulatory system, such as bradycardia and arrhythmia (Ther Adv Drug Saf. 2013 June; 4(3): 119-124).

The most commonly reported side effects are nasopharyngitis, headaches, and fatigue, and more frequently occurring side effects are flu, diarrhea, back pain, increased liver enzyme levels, and colds. The most problematic side effects are a decrease in heart rate, AV conduction block, a mild increase in blood pressure, macular edema, and increased liver enzyme levels during initial treatment. Previous clinical trials have reported that treatment with FTY720 results in bradycardia in the first 24 hours of treatment.

This occurs because FTY720 causes side effects such as bradycardia by acting non-selectively on S1P receptor subtypes (S1PR1, S1PR2, S1PR3, S1PR4, and S1PR5), which can be confirmed through animal experiments using S1P receptor subtype-selective agonists, patch clamp experiments using selective inhibitors, and knockout animal models. Accordingly, it became necessary to develop a compound with greater selectivity for an SIP receptor.

Therefore, while diligently trying to develop an independent compound selectively acting on an SIP subtype receptor among sphingolipid compounds, the inventors confirmed that the compound according to the present invention specifically binds to S1PR1 and S1PR4 receptors to act as a functional antagonist and exhibits an excellent effect compared to conventional specific inhibitors. Thus, the present invention was completed.

### SUMMARY OF THE INVENTION

The present invention is directed to providing a novel compound that has excellent binding affinity to S1PR1 and S1PR4 receptors, a use thereof, and a method of preparing the same.

According to an aspect of the present invention, there is provided a compound represented by the following Formula 1:

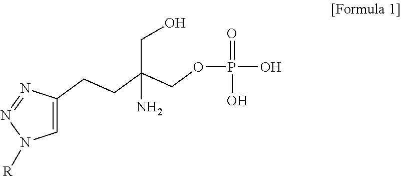

Here, R is C₁₋₁₅ alkyl.

The compound represented by Formula 1 may be any one compound selected from the following compounds:
(1) 2-amino-4-(1-hexyl-1H-1,2,3-triazol-4-yl)-2-(hydroxymethyl)butyl dihydrogen phosphate;
(2) 2-amino-2-(hydroxymethyl)-4-(1-octyl-1H-1,2,3-triazol-4-yl)butyl dihydrogen phosphate;
(3) 2-amino-2-(hydroxymethyl)-4-(1-nonyl-1H-1,2,3-triazol-4-yl)butyl dihydrogen phosphate;
(4) 2-amino-4-(1-decyl-1H-1,2,3-triazol-4-yl)-2-(hydroxymethyl)butyl dihydrogen phosphate;
(5) 2-amino-2-(hydroxymethyl)-4-(1-undecyl-1H-1,2,3-triazol-4-yl)butyl dihydrogen phosphate; and
(6) 2-amino-4-(1-dodecyl-1H-1,2,3-triazol-4-yl)-2-(hydroxymethyl)butyl dihydrogen phosphate.

The compound represented by Formula 1 may be a compound represented by Formula 1a below.

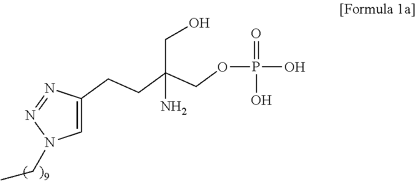

According to another aspect of the present invention, there is provided an S1PR1 and S1PR4 antagonist represented by Formula 1 below:

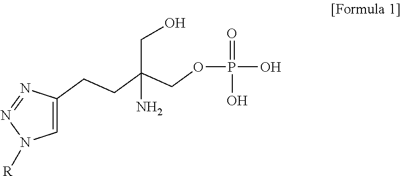

Here, R is C₁₋₁₅ alkyl.

In the present invention, the compound represented by Formula 1 may have at least a 10-fold increase in S1PR1 and S1PR4 binding affinity compared to a compound represented by Formula 2 below:

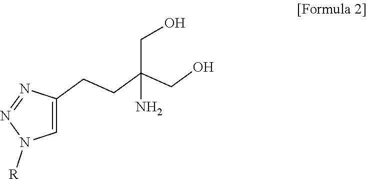

Here, R is C₁₋₁₅ alkyl.

According to still another aspect of the present invention, there is provided a pharmaceutical composition for preventing or treating inflammatory bowel disease, which includes the above compound or a pharmaceutically acceptable salt thereof as an active ingredient.

In the present invention, the inflammatory bowel disease may be ulcerative colitis (UC) or Crohn's disease (CD).

According to yet another aspect of the present invention, there is provided a pharmaceutical composition for preventing or treating alopecia areata (AA), which includes the above compound or a pharmaceutically acceptable salt thereof as an active ingredient.

According to yet another aspect of the present invention, there is provided a pharmaceutical composition for preventing or treating an autoimmune disease, which includes the above compound or a pharmaceutically acceptable salt thereof as an active ingredient.

In the present invention, the autoimmune disease may be one or more selected from the group consisting of asthma, type I diabetes, rheumatoid arthritis, polymyalgia rheumatica, ankylosing spondylitis, psoriatic arthritis, multiple sclerosis (MS), interleukin-17 (IL-17) induced dementia, peripheral neuritis, uveitis, autoimmune cytopenia, autoimmune myocarditis, primary cirrhosis, xeroma, fibromyalgia, Goodpasture's syndrome, autoimmune meningitis, Sjogren's syndrome, Addison's disease, alopecia areata (AA), autoimmune hepatitis, autoimmune parotitis, epididymitis, glomerulonephritis, Grave's disease, celiac disease, Guillain-Barr syndrome, Hashimoto's disease, hemolytic anemia, myasthenia gravis, amyotrophic lateral sclerosis, sarcoidosis, spondyloarthropathies, thyroiditis, vasculitis, myxedema, pernicious anemia, antiphospholipid syndrome, post transplantation late and chronic solid organ rejection, and graft-versus-host disease.

In the present invention, the pharmaceutical composition may further include one or more types of pharmaceutically acceptable carriers, diluents, and/or excipients.

In the present invention, the pharmaceutical composition may not cause cardiovascular disease side effects.

In the present invention, the pharmaceutical composition may be administered intraperitoneally.

According to yet another aspect of the present invention, there is provided a method of preparing a compound represented by Formula 1 below, which includes the following steps:

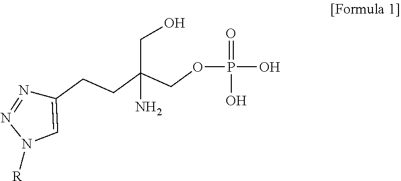

Here, R is C₁₋₁₅ alkyl.
(a) synthesizing a compound represented by Formula 3 below by reacting a compound represented by Formula 2 below with sodium bicarbonate and benzyl chloroformate;

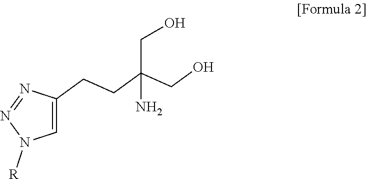

Here, R is C₁₋₁₅ alkyl

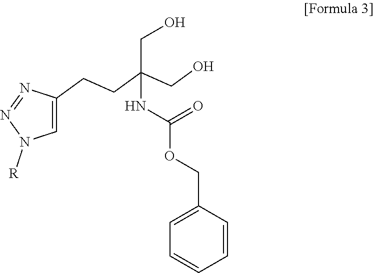

Here, R is C₁₋₁₅ alkyl
(b) synthesizing a compound represented by Formula 4 below by reacting the compound represented by Formula 3 with tetrabenzyl pyrophosphate; and

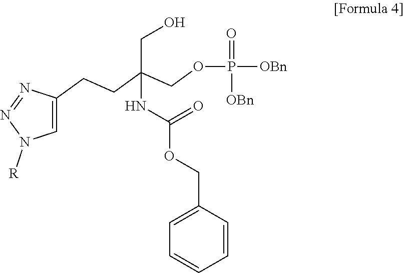

Here, R is C₁₋₁₅ alkyl
(c) synthesizing the compound represented by Formula 1 by reacting the compound represented by Formula 4 with hydrogen (H₂).

In the present invention, in (b), the reaction may be performed by adding diisopropylethylamine and titanium (IV) butoxide.

In the present invention, in (c), the reaction may be performed in the presence of a palladium/carbon catalyst.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows the results of confirming the affinity to S1PR1, S1PR2, S1PR3, and S1PR4 by measuring the degree of arrestin binding of a compound (Compound 1a) according to the present invention;

FIG. 2 shows the results of confirming the pharmacokinetics in plasma after intraperitoneal administration (10 mg/kg) of a compound (Compound 1a) according to the present invention to a mouse;

FIG. 3 shows the results of confirming the lymphocyte % among white blood cells after intraperitoneal administration (10 mg/kg) of a compound (Compound 1a) according to the present invention to a mouse;

FIG. 4 shows the results of confirming the area under the curve (AUC) of a disease activity index after oral administration (5 mg/kg and 10 mg/kg) of a compound (Compound 1a) according to the present invention to DSS-induced colitis mice; and

FIG. 5 shows the results of confirming the colon length recovery effect after oral administration (5 mg/kg and 10 mg/kg) of a compound (Compound 1a) according to the present invention to DSS-induced colitis mice.

### DETAILED DESCRIPTION OF EXEMPLARY EMBODIMENTS

Hereinafter, the present invention will be described in detail.

As a result of the present inventors attempting to develop a novel compound that specifically binds to S1PR1 and S1PR4 receptors and acts as a functional antagonist, it was confirmed that a compound represented by Formula 1 below specifically binds to S1PR1 and S1PR4 receptors with high affinity approximately 300 to 1000 fold higher than a conventional compound represented by Formula 2 below, which is known to be a functional antagonist specifically binding to S1PR1 and S1PR4 receptors, and can act as a functional antagonist.

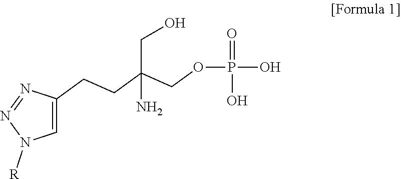

Here, R is C₁₋₁₅ alkyl.

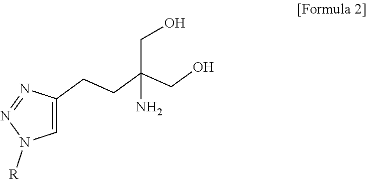

Here, R is C₁₋₁₅ alkyl.

Accordingly, in one aspect, the present invention relates to a novel compound represented by Formula 1 below:

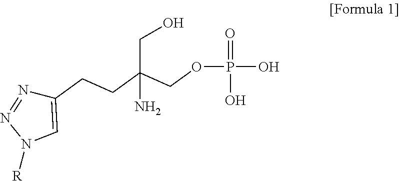

Here, R is C₁₋₁₅ alkyl.

In the present invention, the compound represented by Formula 1 may be any one compound selected from the group consisting of the following compounds:
(1) 2-amino-4-(1-hexyl-1H-1,2,3-triazol-4-yl)-2-(hydroxymethyl)butyl dihydrogen phosphate;
(2) 2-amino-2-(hydroxymethyl)-4-(1-octyl-1H-1,2,3-triazol-4-yl)butyl dihydrogen phosphate
(3) 2-amino-2-(hydroxymethyl)-4-(1-nonyl-1H-1,2,3-triazol-4-yl)butyl dihydrogen phosphate;
(4) 2-amino-4-(1-decyl-1H-1,2,3-triazol-4-yl)-2-(hydroxymethyl)butyl dihydrogen phosphate;
(5) 2-amino-2-(hydroxymethyl)-4-(1-undecyl-1H-1,2,3-triazol-4-yl)butyl dihydrogen phosphate; and
(6) 2-amino-4-(1-dodecyl-1H-1,2,3-triazol-4-yl)-2-(hydroxymethyl)butyl dihydrogen phosphate.

Preferably, the compound represented by Formula 1 may be a compound represented by Formula 1a below, which is 2-amino-4-(1-decyl-1H-1,2,3-triazol-4-yl)-2-(hydroxymethyl)butyl dihydrogen phosphate.

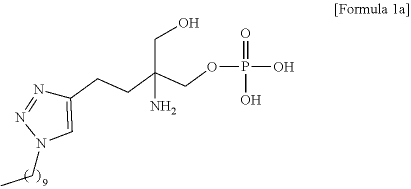 [SMILES: CCn1cc(CCC(N)(CO)COP(=O)(O)O)nn1]

In addition, in another aspect, the present invention relates to an S1PR1 and S1PR4 antagonist represented by Formula 1 below:

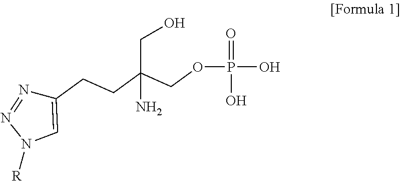

Here, R is C₁₋₁₅ alkyl.

In the present invention, the compound of Formula 1 may have at least a 10-fold increase in S1PR1 and S1PR4 binding affinity compared to a compound represented by Formula 2 below:

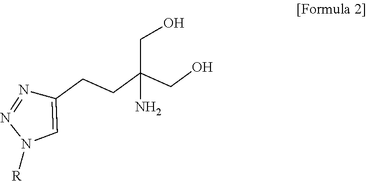

Here, R is C₁₋₁₅ alkyl.

In one embodiment, a compound represented by Formula 1a below according to the present invention may have at least a 10-fold increase in S1PR1 and S1PR4 binding affinity compared to a compound represented by Formula 2a below:

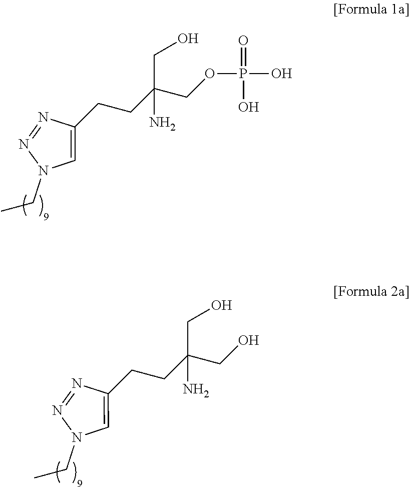

In the present invention, “binding affinity” may be expressed as the ability to bind to S1PR1 and/or S1PR4, and the activity of inhibiting the in vivo function of the receptor by binding to S1PR1 and/or S1PR4.

In one embodiment, the binding activity may be a value compared with EC₅₀ in a competitive inhibition reaction against S1P.

In another embodiment, the binding affinity may be the ability to recruit β-arrestin by activating S1PR1 and/or S1PR4, and β-arrestin recruitment may be measured by analyzing the increase in fluorescence expression by fluorescently labeling β-arrestin.

The compound represented by Formula 1 (e.g., Formula 1a) according to the present invention may activate S1PR1 and/or S1PR4 such that the ability to recruit β-arrestin (i.e., fluorescence expression intensity) may be increased approximately 300 to 1000 times compared to the compound represented by Formula 2 (e.g., Formula 2a).

In the present invention, the compound represented by Formula 1 (e.g., Formula 1a) may have an S1PR1 binding affinity, which is increased approximately 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 times or higher compared to the compound represented by Formula 2 (e.g., Formula 2a).

That is, in the present invention, the compound represented by Formula 1 (e.g., Formula 1a) may have an EC₅₀ in a competitive inhibition reaction with S1P for S1PR1, which is approximately 1/10, 1/20, 1/30, 1/40, 1/50, 1/60, 1/70, 1/80, 1/90, 1/100, 1/110, 1/120, 1/130, 1/140, 1/150, 1/160, 1/170, 1/180, 1/190, 1/200, 1/210, 1/220, 1/230, 1/240, 1/250, 1/260, 1/270, 1/280, 1/290, 1/300, 1/310, 1/320, 1/330, 1/340, 1/350, 1/360, 1/370, 1/380, 1/390, 1/400, 1/410, 1/420, 1/430, 1/440, 1/450, 1/460, 1/470, 1/480, 1/490, 1/500, 1/510, 1/520, 1/530, 1/540, 1/550, 1/560, 1/570, 1/580, 1/590, 1/600, 1/610, 1/620, 1/630, 1/640, 1/650, 1/660, 1/670, 1/680, 1/690, 1/700, 1/710, 1/720, 1/730, 1/740, 1/750, 1/760, 1/770, 1/780, 1/790, 1/800, 1/810, 1/820, 1/830, 1/840, 1/850, 1/860, 1/870, 1/880, 1/890, 1/900, 1/910, 1/920, 1/930, 1/940, 1/950, 1/960, 1/970, 1/980, 1/990, or 1/1000 or lower than that of the compound represented by Formula 1 (e.g., Formula 2a).

In the present invention, the compound represented by Formula 1 (e.g., Formula 1a) may have an S1PR4 binding affinity, which is increased approximately 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, or 370 times or higher compared to the compound represented by Formula 2 (e.g., Formula 2a).

That is, in the present invention, the compound represented by Formula 1 (e.g., Formula 1a) may have an EC₅₀ in a competitive inhibition reaction with S1P for S1PR4, which is approximately 1/10, 1/20, 1/30, 1/40, 1/50, 1/60, 1/70, 1/80, 1/90, 1/100, 1/110, 1/120, 1/130, 1/140, 1/150, 1/160, 1/170, 1/180, 1/190, 1/200, 1/210, 1/220, 1/230, 1/240, 1/250, 1/260, 1/270, 1/280, 1/290, 1/300, 1/310, 1/320, 1/330, 1/340, 1/350, 1/360, or 1/370 or lower than that of the compound represented by Formula 2 (e.g., Formula 2a).

In one aspect, the binding affinity may be evaluated by an EC₅₀ according to a β-arrestin recruitment assay.

In another aspect, the present invention relates to a composition that includes a compound represented by Formula 1 below (e.g., Formula 1a), and more particularly, a composition for inhibiting S1PR1 and S1PR4, which includes a compound represented by Formula 1 below (e.g., Formula 1a):

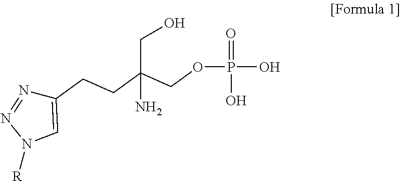

Here, R is C₁₋₁₅ alkyl.

In still another aspect, the present invention relates to a pharmaceutical composition for preventing or treating inflammatory bowel disease, which includes the compound represented by Formula 1 (e.g., Formula 1a) or a pharmaceutically acceptable salt thereof as an active ingredient.

In yet another aspect, the present invention relates to a use of the compound represented by Formula 1 (e.g., Formula 1a) or a pharmaceutically acceptable salt thereof, or the above pharmaceutical composition for preventing or treating inflammatory bowel disease.

In yet another aspect, the present invention relates to the compound represented by Formula 1 (e.g., Formula 1a) or a pharmaceutically acceptable salt thereof or a pharmaceutical composition comprising the compound or the pharmaceutically acceptable salt thereof, for use in the prevention or treatment of inflammatory bowel disease.

In yet another aspect, the present invention relates to a method of preventing or treating inflammatory bowel disease, which includes administering the compound represented by Formula 1 (e.g., Formula 1a) or a pharmaceutically acceptable salt thereof, or the above pharmaceutical composition to a subject in need thereof.

In yet another aspect, the present invention relates to a use of the compound represented by Formula 1 (e.g., Formula 1a) or a pharmaceutically acceptable salt thereof, or the above pharmaceutical composition for preparing a drug for preventing or treating inflammatory bowel disease.

In the present invention, the inflammatory bowel disease may be ulcerative colitis (UC) or Crohn's disease (CD), but the present invention is not limited thereto.

In yet another aspect, the present invention relates to a pharmaceutical composition for preventing or treating alopecia areata (AA), which includes the compound represented by Formula 1 (e.g., Formula 1a) or a pharmaceutically acceptable salt thereof as an active ingredient.

In yet another aspect, the present invention relates to a use of the compound represented by Formula 1 (e.g., Formula 1a) or a pharmaceutically acceptable salt thereof or the above pharmaceutical composition for preventing or treating alopecia areata (AA).

In yet another aspect, the present invention relates to the compound represented by Formula 1 (e.g., Formula 1a) or a pharmaceutically acceptable salt thereof or a pharmaceutical composition comprising the compound or the pharmaceutically acceptable salt thereof, for use in the prevention or treatment of alopecia areata (AA).

In yet another aspect, the present invention relates to a method of preventing or treating alopecia areata (AA), which includes administering the compound represented by Formula 1 (e.g., Formula 1a) or a pharmaceutically acceptable salt thereof or the above pharmaceutical composition to a subject in need thereof.

In yet another aspect, the present invention relates to a use of the compound represented by Formula 1 (e.g., Formula 1a) or a pharmaceutically acceptable salt thereof or the above pharmaceutical composition for preparing a drug for preventing or treating alopecia areata (AA).

In yet another aspect, the present invention relates to a pharmaceutical composition for preventing or treating an autoimmune disease, which includes the compound represented by Formula 1 (e.g., Formula 1a) or a pharmaceutically acceptable salt thereof as an active ingredient.

In yet another aspect, the present invention relates to a use of the compound represented by Formula 1 (e.g., Formula 1a) or a pharmaceutically acceptable salt thereof or the above pharmaceutical composition for preventing or treating an autoimmune disease.

In yet another aspect, the present invention relates to the compound represented by Formula 1 (e.g., Formula 1a) or a pharmaceutically acceptable salt thereof or a pharmaceutical composition comprising the compound or the pharmaceutically acceptable salt thereof, for use in the prevention or treatment of an autoimmune disease.

In yet another aspect, the present invention relates to a method of preventing or treating an autoimmune disease, which includes administering the compound represented by Formula 1 (e.g., Formula 1a) or a pharmaceutically acceptable salt thereof or the above pharmaceutical composition to a subject in need thereof.

In yet another aspect, the present invention relates to a use of the compound represented by Formula 1 (e.g., Formula 1a) or a pharmaceutically acceptable salt thereof for preparing a drug for preventing or treating an autoimmune disease.

In the present invention, the autoimmune disease may be one or more selected from the group consisting of asthma, type I diabetes, rheumatoid arthritis, polymyalgia rheumatica, ankylosing spondylitis, psoriatic arthritis, multiple sclerosis (MS), interleukin-17 (IL-17) induced dementia, peripheral neuritis, uveitis, autoimmune cytopenia, autoimmune myocarditis, primary cirrhosis, xeroma, fibromyalgia, Goodpasture's syndrome, autoimmune meningitis, Sjogren's syndrome, Addison's disease, alopecia areata (AA), autoimmune hepatitis, autoimmune parotitis, epididymitis, glomerulonephritis, Grave's disease, celiac disease, Guillain-Barr syndrome, Hashimoto's disease, hemolytic anemia, myasthenia gravis, amyotrophic lateral sclerosis, sarcoidosis, spondyloarthropathies, thyroiditis, vasculitis, myxedema, pernicious anemia, antiphospholipid syndrome, post transplantation late and chronic solid organ rejection, and graft-versus-host disease, but the present invention is not limited thereto.

In yet another aspect, the present invention relates to a pharmaceutical composition for preventing or treating focal segmental glomerulosclerosis (FSGS), which includes the compound represented by Formula 1 (e.g., Formula 1a) or a pharmaceutically acceptable salt thereof as an active ingredient.

In yet another aspect, the present invention relates to a use of the compound represented by Formula 1 (e.g., Formula 1a) or a pharmaceutically acceptable salt thereof or the above pharmaceutical composition for preventing or treating focal segmental glomerulosclerosis (FSGS).

In yet another aspect, the present invention relates to the compound represented by Formula 1 (e.g., Formula 1a) or a pharmaceutically acceptable salt thereof or a pharmaceutical composition comprising the compound or the pharmaceutically acceptable salt thereof, for use in the prevention or treatment of focal segmental glomerulosclerosis (FSGS).

In yet another aspect, the present invention relates to a method of preventing or treating focal segmental glomerulosclerosis (FSGS), which includes administering the compound represented by Formula 1 (e.g., Formula 1a) or a pharmaceutically acceptable salt thereof or the above pharmaceutical composition to a subject in need thereof.

In yet another aspect, the present invention relates to a use of the compound represented by Formula 1 (e.g., Formula 1a) or a pharmaceutically acceptable salt thereof or the above pharmaceutical composition for preparing a drug for preventing or treating focal segmental glomerulosclerosis (FSGS).

In yet another aspect, the present invention relates to a pharmaceutical composition for preventing or treating interstitial fibrosis and tubular atrophy (IFTA), which includes the compound represented by Formula 1 (e.g., Formula 1a) or a pharmaceutically acceptable salt thereof as an active ingredient.

In yet another aspect, the present invention relates to a use of the compound represented by Formula 1 (e.g., Formula 1a) or a pharmaceutically acceptable salt thereof or the above pharmaceutical composition for preventing or treating interstitial fibrosis and tubular atrophy (IFTA).

In yet another aspect, the present invention relates to the compound represented by Formula 1 (e.g., Formula 1a) or a pharmaceutically acceptable salt thereof or a pharmaceutical composition comprising the compound or the pharmaceutically acceptable salt thereof, for use in the prevention or treatment of interstitial fibrosis and tubular atrophy (IFTA).

In yet another aspect, the present invention relates to a method of preventing or treating interstitial fibrosis and tubular atrophy (IFTA), which includes administering the compound represented by Formula 1 (e.g., Formula 1a) or a pharmaceutically acceptable salt thereof or the above pharmaceutical composition to a subject in need thereof.

In yet another aspect, the present invention relates to a use of the compound represented by Formula 1 (e.g., Formula 1a) or a pharmaceutically acceptable salt thereof or the above pharmaceutical composition for preparing a drug for preventing or treating interstitial fibrosis and tubular atrophy (IFTA).

In the present invention, the pharmaceutical composition may further include one or more pharmaceutically acceptable carriers, diluents, and/or excipients.

In the present invention, the compound represented by Formula 1 (e.g., Formula 1a) or a pharmaceutically acceptable salt thereof may be included at 0.1 to 90 wt % with respect to the total weight of the pharmaceutical composition.

In the present invention, the compound represented by Formula 1 (e.g., Formula 1a) or a pharmaceutically acceptable salt thereof, or the pharmaceutical composition may not cause cardiovascular disease side effects.

In the present invention, the compound represented by Formula 1 (e.g., Formula 1a) or a pharmaceutically acceptable salt thereof, or the above pharmaceutical compositions may be administered intraperitoneally, but the present invention is not limited thereto.

“Prevention” used in the present invention refers to all actions of inhibiting or delaying the occurrence of a target disease by administering a pharmaceutical composition to a subject.

“Treatment” used in the present invention refers to all actions involved in alleviating or beneficially changing symptoms of a target disease by administering a pharmaceutical composition to a subject.

The compound represented by Formula 1 (e.g., Formula 1a) may be used in the form of a pharmaceutically acceptable salt. Here, the salt may be an acid addition salt formed by a pharmaceutically acceptable free acid.

More particularly, the acid addition salt may be obtained from an inorganic acid such as hydrochloric acid, nitric acid, phosphoric acid, sulfuric acid, hydrobromic acid, hydroiodic acid, nitrous acid or phosphorous acid, aliphatic mono- or di-carboxylate, phenyl-substituted alkanoate, hydroxy alkanoate, or alkane dioate, a non-toxic organic acid such as aromatic acid, aliphatic or aromatic sulfonic acid, or an organic acid such as acetic acid, benzoic acid, citric acid, lactic acid, maleic acid, gluconic acid, methanesulfonic acid, 4-toluenesulfonic acid, tartaric acid, or fumaric acid.

In addition, the pharmaceutically non-toxic salt may be a sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxylbenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxylbutyrate, glycholate, malate, tartrate, methanesulfonate, propanesulfonate, naphthalene-1-sulfonate, naphthalene-2-sulfonate, or mandelate.

The acid addition salt may be prepared by a conventional method. For example, a derivative of the compound represented by Formula 1 (e.g., Formula 1a) may be prepared by filtering and drying a precipitate obtained by dissolution in an organic solvent such as methanol, ethanol, acetone, methylene chloride, or acetonitrile, and adding an organic or inorganic acid, or prepared by distilling the solvent and an excess acid under reduced pressure, and drying it under an organic solvent to crystallize.

In addition, a pharmaceutically acceptable metal salt may be prepared using a base. For example, a pharmaceutically acceptable metal salt may be prepared by dissolving the compound represented by Formula 1 (e.g., Formula 1a) in an excess alkali metal hydroxide or alkaline earth metal hydroxide solution, filtering a non-dissolved compound salt, and evaporating and drying the filtrate. As a metal salt, a sodium, potassium, or calcium salt is preferably prepared. In addition, the corresponding salt may be obtained by reacting an alkali metal or alkaline earth metal salt with a suitable silver salt (e.g., silver nitrate).

In addition, the pharmaceutical composition according to the present invention may include not only the compound represented by Formula 1 (e.g., Formula 1a) and a pharmaceutically acceptable salt thereof as an active ingredient, but also a material selected from solvates, optical isomers, and hydrates, which can be prepared therefrom, as an active ingredient.

A pharmaceutical composition that includes the compound represented by Formula 1 (e.g., Formula 1a), an optical isomer thereof, or a pharmaceutically acceptable salt thereof as an active ingredient may be changed to a formulation for oral administration and a different type of formulation exhibiting pharmacological activity.

Formulations for oral administration may be prepared in the form of troches, lozenges, tablets, aqueous suspensions, oily suspensions, powders, granules, emulsions, hard capsules, soft capsules, syrups, or elixirs, but the present invention is not limited thereto.

In addition, to prepare the pharmaceutical composition according to the present invention in a formulation for oral administration, a binder such as lactose, saccharose, sorbitol, mannitol, starch, amylopectin, cellulose, or gelatin; an excipient such as dicalcium phosphate; a disintegrant such as corn starch or sweet potato starch; a lubricant such as magnesium stearate, calcium stearate, sodium stearyl fumarate, or polyethylene glycol wax; a sweetener; a fragrant; or a syrup may be used. In addition, in the case of capsules, in addition to the above-described materials, a liquid carrier such as fatty oil may be further used.

A pharmaceutical composition that includes the compound represented by Formula 1 (e.g., Formula 1a), an optical isomer thereof, or a pharmaceutically acceptable salt thereof as an active ingredient may be used in various formulations suitable for parenteral administration.

A formulation for parenteral administration may include an injection, a suppository, a powder for respiratory inhalation, a spray aerosol, an ointment, a powder for coating, an oil, or a cream, but the present invention is not limited thereto.

In addition, to prepare the pharmaceutical composition according to the present invention in a formulation for parenteral administration, sterilized aqueous solutions, non-aqueous solutions, suspensions, emulsions, freeze-dried preparations, or external preparations may be used. Specifically, non-aqueous solvents and suspensions may include propylene glycol, polyethylene glycol, a vegetable oil such as olive oil, and an injectable ester such as ethyl oleate.

When the pharmaceutical composition according to the present invention is prepared as an injection, the pharmaceutical composition may be mixed in water with a stabilizer or buffer to prepare a solution or suspension and may be prepared for unit administration in ampoules or vials. In addition, when the pharmaceutical composition according to the present invention is prepared as an aerosol, it may be mixed with an additive such as a propellant to disperse a water-dispersed concentrate or wet powder. In addition, when the pharmaceutical composition according to the present invention is prepared as an ointment or cream, animal oil, vegetable oil, wax, paraffin, starch, tragacanth, a cellulose derivative, polyethylene glycol, silicone, bentonite, silica, talc, or zinc oxide may be used as a carrier.

The pharmaceutical composition according to the present invention may further include one or more therapeutics suitable for treating the disease.

One or more other therapeutics suitable for treating the disease may be administered together or separately. When administered separately, they may be administered simultaneously or sequentially in any order. The dosage and administration time for the compound represented by Formula 1 (e.g., Formula 1a) or a pharmaceutically acceptable salt thereof, and a different therapeutic may be selected to achieve desire combination treatment effects.

The pharmaceutical composition according to the present invention may further include a pharmaceutically acceptable carrier. “Pharmaceutically acceptable” means that a compound, which is commonly used in the pharmaceutical field, does not irritate an organism when administered and does not suppress the biological activity and characteristics of the administered compound.

The type of carrier is not particularly limited, and any carrier conventionally used in the art can be used. Non-limiting examples of carriers may include saline, sterile water, Ringer's solution, buffered saline, albumin injectable solutions, lactose, dextrose, sucrose, sorbitol, mannitol, xylitol, erythritol, maltitol, maltodextrin, glycerol, and ethanol. These may be used alone or in combination of two or more thereof.

In addition, as necessary, other pharmaceutically acceptable additives such as an excipient, an antioxidant, a buffer, and a bacteriostatic may be added to the pharmaceutical composition according to the present invention. In addition, as necessary, a filler, an extender, a wetting agent, a disintegrant, a dispersant, a surfactant, a binder, or a lubricant may be additionally added to the pharmaceutical composition according to the present invention.

The pharmacologically effective amount and dosage of the pharmaceutical composition according to the present invention for the human body may vary depending on a preparation method, administration mode, administration time, and/or administration route of the pharmaceutical composition. The dosage may vary according to various factors including the type and degree of reaction to be achieved by administration of the pharmaceutical composition, the type, age, body weight, and general health condition of a subject to which the composition is administered, symptoms or severity of a disease, sex, diet, excretion, and components of other compositions such as drugs used simultaneously or concurrently, and similar factors widely known in the medical field. Those of ordinary skill in the art may easily determine and prescribe an effective dosage for desired treatment.

In addition, the administration route and administration mode of the pharmaceutical composition according to the present invention may each be independent, and its mode is not particularly limited. Any route and mode for administration that allows an active ingredient to reach the desired target area by administering the pharmaceutical composition according to the present invention may be used.

The pharmaceutical composition according to the present invention may be administered by oral or parenteral administration. For example, parenteral administration may include intravenous administration, intraperitoneal administration, intramuscular administration, transdermal administration, or subcutaneous administration. In addition, the pharmaceutical composition according to the present invention may be applied on a diseased area, or sprayed or inhaled, but the present invention is not limited thereto. In addition, the pharmaceutical composition according to the present invention may be used for treatment of the above diseases in a subject in need thereof. Here, the type of subject may be, but is not particularly limited, a mammal, and preferably, a human.

The S1PR1 receptor is mainly present in lymphocytes and plays a role in regulating the release of lymphocytes from immune cells. The S1PR4 receptor is also present in lymphocytes so it plays a role in not only immune cell trafficking but also in T cell regulation, and thus is involved in inflammatory responses.

Accordingly, the compound of the present invention acts as a functional antagonist for the S1PR1 receptor to inhibit the release of lymphocytes, and acts on S1PR4 to exhibit an effective anti-inflammatory effect, resulting in preventing or treating the above diseases.

Specifically, the compound of the present invention acts as a functional antagonist for S1PR1 and S1PR4 through a mechanism of specifically binding to S1PR1 and S1PR4 and then entering cells and eliminating them, thereby exhibiting an effect of preventing or treating the disease. In addition, the pharmaceutical composition according to the present invention may act as a functional antagonist for S1PR1 and S1PR4 so as not to cause cardiovascular disease side effects.

In yet another aspect, the present invention relates to a method of preparing a compound represented by Formula 1 below, which includes the following steps:

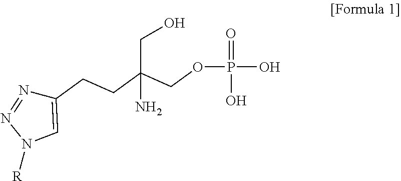

Here, R is C₁₋₁₅ alkyl
(a) synthesizing a compound represented by Formula 3 below by reacting a compound represented by Formula 2 below with sodium bicarbonate and benzyl chloroformate;

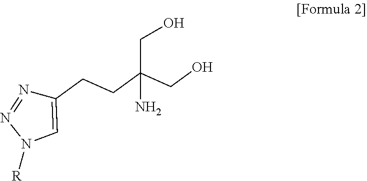

Here, R is C₁₋₁₅ alkyl

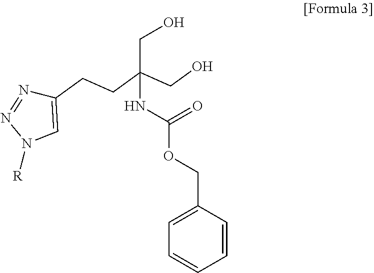

Here, R is C₁₋₁₅ alkyl
(b) synthesizing a compound represented by Formula 4 below by reacting the compound represented by Formula 3 with tetrabenzyl pyrophosphate; and

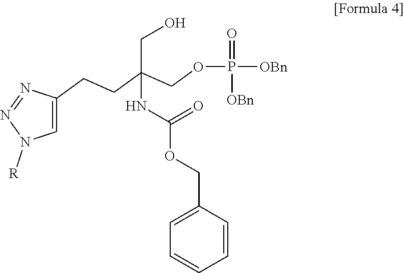

Here, R is C₁₋₁₅ alkyl
(c) synthesizing the compound represented by Formula 1 by reacting the compound represented by Formula 4 with hydrogen (H₂).

In yet another aspect, the present invention relates to a method of preparing a compound represented by Formula 1a below, which includes the following steps:

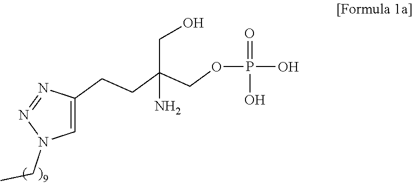

(a) synthesizing a compound represented by Formula 3a below by reacting a compound represented by Formula 2a below with sodium bicarbonate and benzyl chloroformate;

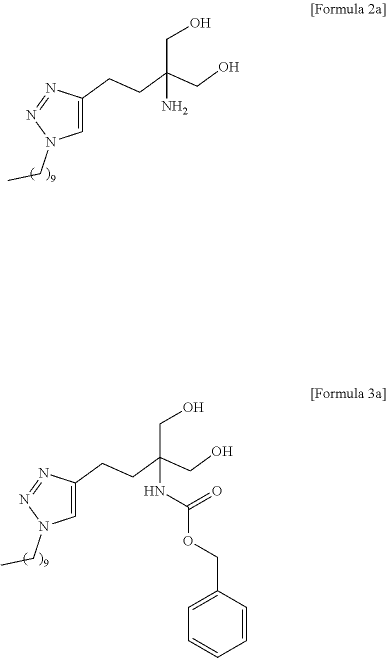

(b) synthesizing a compound represented by Formula 4a below by reacting the compound represented by Formula 3a with tetrabenzyl pyrophosphate; and

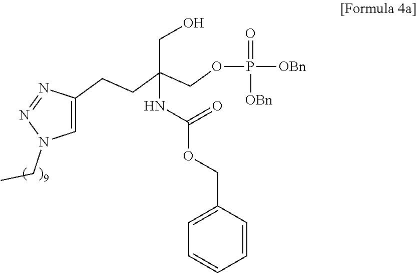

(c) synthesizing the compound represented by Formula 1a by reacting the compound represented by Formula 4a with hydrogen (H₂).

In the present invention, in (b), the reaction may be performed by adding diisopropylethylamine and titanium (IV) butoxide.

In the present invention, in (c), the reaction may be performed in the presence of a palladium/carbon catalyst.

Hereinafter, the present invention will be described in more detail with reference to examples. The examples are merely provided to more fully describe the present invention, and it will be obvious to those of ordinary skill in the art that the scope of the present invention is not limited by the following examples.

### Preparation Example

### Step 1) Synthesis of benzyl (4-(1decyl-1H-1,2,3-triazol-4-yl)-1-hydroxy-2-(hydroxymethyl)butan-2-yl)carbamate (3a)

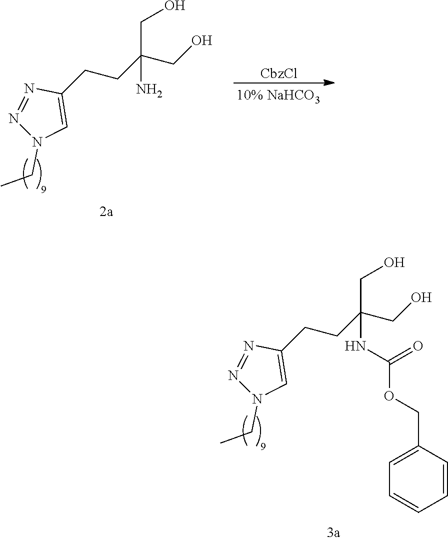

2.04 g of Compound 2a (a compound represented by Formula 2, wherein R is C₁₀ alkyl, refer to Korean Patent Publication No. 10-1830244), 525 mg of sodium bicarbonate, and 50 mL of distilled water were added to 90 mL of ethyl acetate and stirred at room temperature for 30 minutes. 1.6 g of benzyl chloroformate was slowly added at room temperature. After stirring at room temperature for 6 hours, 3 mL of methanol was added and stirred for 30 minutes to achieve layer separation. An organic layer was washed with 20 mL of a saturated saline solution, dried with sodium sulfate, filtered, and then concentrated under reduced pressure. The concentrate was separated by column chromatography, thereby obtaining 1.87 g of Compound 3a.

1H NMR (400 MHz, CDCl3) δ 7.21˜7.33 (m, 5H), 7.18 (s, 1H), 4.98 (br s, 2H), 4.25˜4.28 (m, 2H), 4.02˜4.15 (m, 4H), 3.48 (m, 2H), 2.65˜2.62 (m, 2H), 1.92 (m, 2H), 1.80 (m, 2H), 1.28 (m, 14H), 0.86 (t, J=8.0 Hz, 3H).

LC-MS [M+H]+=461.

### Step 2) Synthesis of benzyl (1-((bis(benzyloxy)phosphoryl)oxy)-4-(1-decyl-1H-1,2,3-triazol-4-yl)-2-(hydroxymethyl)butan-2-yl)carbamate (4a)

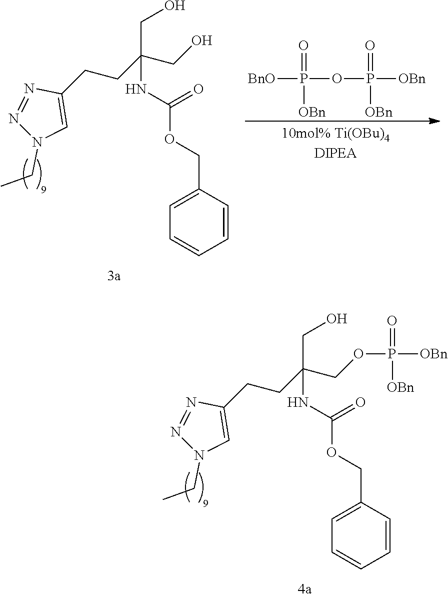

1.87 g of Compound 3a and 4.24 mL of N,N-diisopropylethylamine were added to 50 mL of tetrahydrofuran and stirred at room temperature for 30 minutes. 2.84 g of tetrabenzyl pyrophosphate and 64.48 mg of titanium (IV) butoxide were sequentially added and then stirred at room temperature overnight. 20 mL of a saturated sodium bicarbonate solution and 30 mL of ethyl acetate were added to the reaction solution and stirred for 10 minutes to achieve layer separation. An organic layer was washed with 30 mL of a saturated saline solution, dried with sodium sulfate, filtered, and then concentrated under reduced pressure. The concentrate was separated by column chromatography, thereby obtaining 2.14 g of Compound 4a.

1H NMR (400 MHz, CDCl3) δ 7.21˜7.33 (m, 15H), 7.21 (s, 1H), 5.00˜5.24 (s, 6H), 4.25˜4.28 (m, 2H), 4.07˜4.17 (m, 2H), 3.52 (m, 2H), 2.65˜2.69 (m, 2H), 2.23˜2.38 (m, 2H), 1.91˜1.95 (m, 2H), 1.85˜1.87 (m, 2H), 1.30 (m, 14H), 0.88 (t, J=8.0 Hz, 3H).

LC-MS [M+H]+=721.

### Step 3) Synthesis of 2-amino-4-(1-decyl-1H-1,2,3-triazol-4-yl)-2-(hydroxymethyl)butyl dihydrogen phosphate (1a)

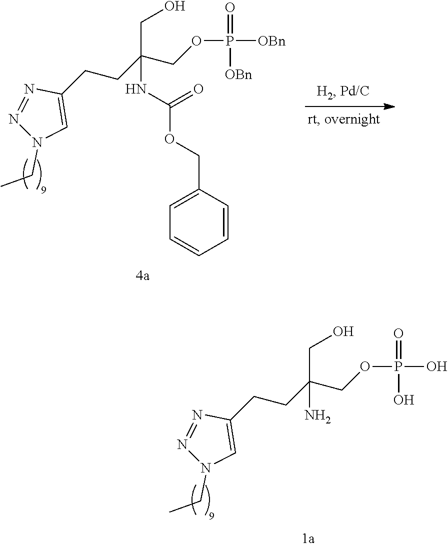

2.14 g of Compound 4a was added to 50 mL of methanol and dissolved, and then bubbled with nitrogen for 10 minutes. 0.8 g of 10% palladium/carbon was added and reacted using a hydrogen balloon at room temperature overnight. After filtration with a microfilter and concentration under reduced pressure, 20 mL of an EA/Hexane=1/4 solution was added to the concentration residue, stirred for 1 hour, filtered and dried, thereby obtaining 0.82 g of Compound 1a, (a compound represented by Formula 1, wherein R is C₁₀ alkyl).

1H NMR (400 MHz, DMSO-d6) δ 7.86 (s, 1H), 4.25˜4.28 (m, 2H), 3.85 (d, 2H), 3.47˜3.64 (dd, 2H), 2.67˜2.70 (m, 2H), 1.90˜1.93 (m, 2H), 1.75˜1.78 (m, 2H), 1.34 (m, 14H), 0.89 (t, J=8.0 Hz, 3H).

LC-MS [M+H]+=407.

### Experimental Example 1. Arrestin Translocation Assay

### [Formula 1a]2-amino-4-(1-decyl-1H-1,2,3-triazol-4-yl)-2-(hydroxymethyl)butyl dihydrogen phosphate

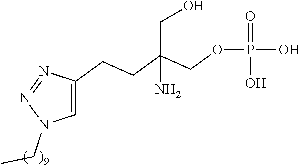

### [Formula 2a]2-amino-2-(2-(1-decyl-1H-1,2,3-triazol-4-yl)ethyl)propan-1,3-diol

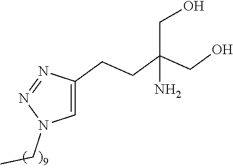

The affinity to S1PR1, S1PR2, S1PR3, and S1PR4 of the compound of the present invention, Compound 1a (a compound represented by Formula 1, wherein R is C₁₀ alkyl), and the comparative example, Compound 2a (a compound represented by Formula 2, wherein R is C₁₀ alkyl), was compared through β-arrestin recruitment assay.

To this end, each of the cell lines in which S1PR1, S1PR2, S1PR3, and S1PR4 are overexpressed (PathHunter® CHO-K1 eXpress β-Arrestin GPCR cells; DiscoverX, Fremont, USA) was seeded in a 384-well plate at 5000 cells/well and cultured using an AssayComplete™ Cell Culture Kit-107 (DiscoverX, Fremont, USA). Compound 1a or Compound 2a was diluted by 1/3 at 10 μM and treated at 10 points. A PathHunter detection reagent cocktail (DiscoverX, Fremont, USA) was added in accordance with the manufacturer's instructions and incubated for 1 hour, followed by measuring a chemiluminescent signal (luminescence reader, 0.1 to 1 second/well).

The types and affinities of control materials are listed in Table 1.

TABLE 1

| Assay format | Reference cpd | Target | EC₅₀ | |
|---|---|---|---|---|
| Agonism | S1P | S1PR1 | 16.3 | nM |
| | | S1PR2 | 17.6 | nM |
| | | S1PR3 | 59.2 | nM |
| | | S1PR4 | 229.3 | nM |

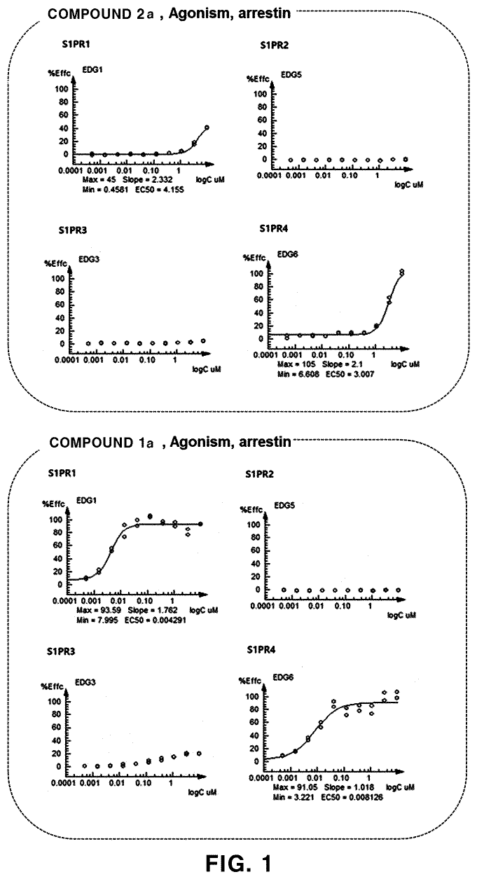

The results of evaluating the agonism affinity to each subtype of S1PR of Compound 1a (a compound represented by Formula 1, wherein R is C₁₀ alkyl) and Compound 2a (a compound represented by Formula 2, wherein R is C₁₀ alkyl) are shown in Table 2 below and FIG. 1. Specifically, it was confirmed that Compound 1a and Compound 2a exhibited selective affinity to both S1PR1 and S1PR4, and particularly, Compound 1a exhibited an affinity approximately 300 to 1000 times higher than that of Compound 2a (EC₅₀ for S1PR1: 4.3 nM vs 4.2 μM, EC₅₀ for S1PR4: 8.1 nM vs 3.0 μM).

TABLE 2

| Cpd. | Target | EC₅₀ | | Max response (%) |
|---|---|---|---|---|
| Compound 2a | S1PR1 | 4.2 | uM | 41.0 |
| | S1PR2 | >10 | uM | 4.4 |
| | S1PR3 | >10 | uM | 0.0 |
| | S1PR4 | 3.0 | uM | 101.3 |
| Compound 1a | S1PR1 | 4.3 | nM | 92.8 |
| | S1PR2 | >10 | uM | 0 |
| | S1PR3 | >10 | uM | 0 |
| | S1PR4 | 8.1 | nM | 102.0 |

### Experimental Example 2. Pharmacokinetic and Pharmacodynamic Evaluation of Compound 1a Upon Intraperitoneal Administration to Mice

To analyze the pharmacokinetics of Compound 1a, 10 mg/kg of Compound 1a dissolved in distilled water was administered intraperitoneally once to ICR mice (DBL; n=12), and then 100 μL of blood was collected a total of 10 times at 0.083, 0.25, 0.5, 1, 2, 4, 6, 8, 12, and 24 hours. The collected blood was mixed well in a heparin (5 IU/mL)-treated tube and centrifuged using a centrifuge (MF 300, Hanil Scientific) at 4° C. and 5000 rpm for 10 minutes to separate the plasma, and the separated plasma was dispensed in a labeled tube at approximately 30 to 40 μL/tube and stored in an ultra-low temperature freezer (approximately −80° C.). Afterward, the plasma samples were analyzed using LC-MS/MS (HPLC: Agilent 1200 Series (Agilent, USA)/MS/MS: AB Sciex 4000 QTrap (AB SCIEX, USA).

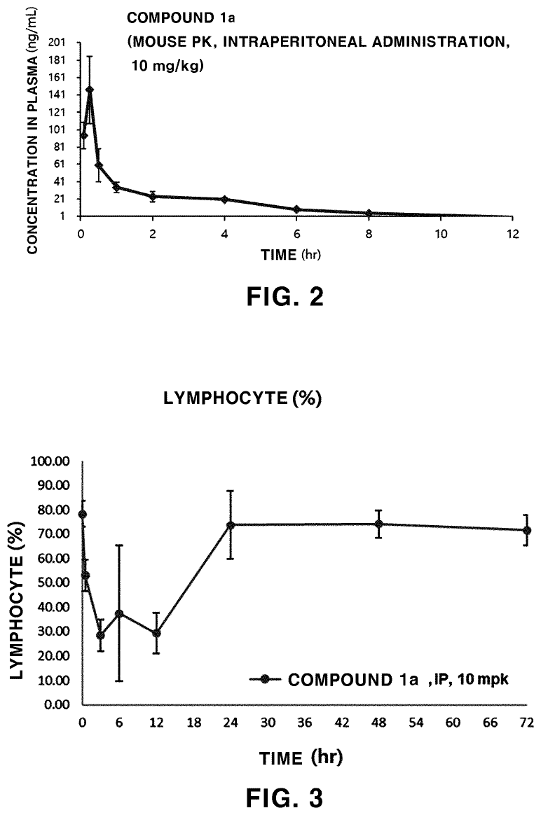

Drug concentrations over time upon the intraperitoneal administration of Compound 1a are shown in Table 3, and pharmacokinetic data is shown in Table 4 and FIG. 2. Particularly, the Cmax of Compound 1a was determined to be approximately 147 ng/mL, and its AUC was determined to be approximately 185 ng-hr/mL.

TABLE 3

| Time | Compound 1a (IP, 10 mg/kg) |
|---|---|
| (hr) | Compound 1a (ng/mL) |
| 0.083 | 94.38 |
| 0.25 | 146.82 |
| 0.5 | 59.85 |
| 1 | 34.24 |
| 2 | 23.73 |
| 4 | 20.56 |
| 6 | 8.77 |
| 8 | 4.48 |
| 12 | ND |
| 24 | ND |

TABLE 4

| | | Compound, dose |
|---|---|---|
| | | Compound 1a, 10 mg/kg, |
| | | Route |
| | | IP |
| PK Parameters | Units | Compound 1a |
| AUCₗₐₛₜ | h*ng/mL | 184.5 |
| AUC_{INF}_{_{—}}obs | h*ng/mL | 196.3 |
| Cₘₐₓ | ng/mL | 146.8 |
| Tₘₐₓ | h | 0.3 |
| t_{1/2} | h | 1.8 |

Meanwhile, to analyze the pharmacodynamics of Compound 1a, 10 mg/kg of the Compound 1a dissolved in distilled water was administered intraperitoneally to ICR mice (DBL; n=3), and 100 μL of blood was collected a total of 8 times at 0.5, 3, 6, 8, 12, 24, 48, and 72 hours and then WBCs (5 differential counts) were measured using an automated hematology analyzer (Advia 2020i, SIEMENS).

As a result, as shown in FIG. 3, the lymphocyte % among white blood cells (WBCs) was 28.5±6.5% at 3 hours after the administration of Compound 1a, which is more than 50% lower than that before the administration of Compound 1a (78.4±5.3%), and 29.2±8.3% at 12 hours after the administration of Compound 1a, confirming that the decrease in lymphocytes was maintained up to 12 hours and restored to the initial percentage at 24 hours.

### Experimental Example 3. Confirmation of Inhibitory Efficacy on Inflammatory Bowel Disease Using Mouse DSS-Induced Model

The effectiveness of Compound 1a on inflammatory bowel disease was evaluated using dextran sulfate sodium (DSS) induced colitis models.

In the mouse model, inflammatory bowel disease was induced by administering 2.5% DSS to 6- to 10-week-old female C57BL/6 mice (Koatech). The administration groups consisted of a test group to which Compound 1a was administered, a vehicle group [5% DW, 5% Tween 20, 90% 0.1N HCl], and a normal control, and each group had 10 animals. The compound of the present invention, Compound 1a, was orally administered to the inflammatory bowel disease-induced models at 5 and 10 mg/kg once a day for 7 days.

The disease activity index (DAI) of the DSS-induced colitis mice and the areas under the curve (AUC) of the DAI thereof were confirmed. DAI represents the total score of weight loss relative to the initial weight of a mouse, stool consistency, and the degree of intestinal bleeding. The calculation criteria for DAI are shown in Table 5 below, and DAI was evaluated on day 0, 1, 2, 3, 4, 5, 6, and 7.

TABLE 5

| | Weight loss | | |
|---|---|---|---|
| | to initial | | |
| Score | weight | Stool consistency | Intestinal bleeding |
| 0 | none | Normal | Negative hemoccult |
| 1 | 1-5% | Soft but still formed | Positive hemoccult |
| | | | (weak luminol intensity) |
| 2 | 6-10% | Soft | Positive hemoccult |
| | | | (strong luminol intensity) |
| 3 | 11-18% | Very soft; wet | Visual confirmation of blood |
| | | | in stool |
| 4 | >18% | Watery diarrhea | Intestinal bleeding |

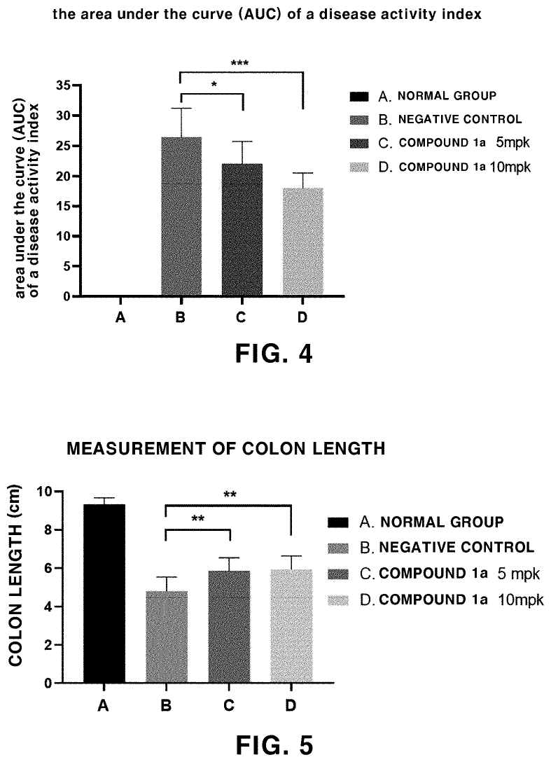

The experimental results can confirm statistically significant effects of DAI reduction in both groups to which 5 mg/kg of Compound 1a and 10 mg/kg of Compound 1a were administered, compared to the negative control (Vehicle). Particularly, as a result of administering 10 mg/kg of Compound 1a, it can be confirmed that the mice exhibited significant effects on clinical indicators such as weight loss, stool consistency, and the degree of intestinal bleeding (FIG. 4).

Meanwhile, the colon tissue of a DSS-induced colitis mouse was collected, and its length was measured. Colon length measurement indicates whether damage to the colon tissue of the mouse is reduced and the extent to which the colon length is maintained, and the colon length measurement was evaluated after the completion of the administration of a positive control material and a test material.

As a result of the experiment, a statistically significant increase in colon length was confirmed in both groups to which 5 mg/kg of Compound 1a and 10 mg/kg of Compound 1a were administered compared to the negative control (Vehicle). Particularly, as a result of administering 5 mg/kg of Compound 1a and 10 mg/kg of Compound 1a, significant results were confirmed in the reduction in damage to colon tissue and the maintenance of a colon length (FIG. 5).

The above evaluation results were subjected to statistical analysis between the negative control and the test group or between two test groups. For comparison between groups, the independent t-test or repeated measures ANOVA was used, and the p value was significant at 0.05.

As a result, it was confirmed that Compound 1a is a material that exhibits an inhibitory effect on inflammatory bowel disease.

A compound represented by Formula 1 according to the present invention has significantly improved binding strength to S1PR1 and S1PR4 compared to a compound represented by Formula 2, known as a functional antagonist for S1PR1 and S1PR4, thus exhibiting an excellent effect on the prevention or treatment of inflammatory bowel disease, alopecia areata (AA), or an autoimmune disease.

What is claimed is:

1. A compound represented by Formula 1 below or a pharmaceutically acceptable salt thereof: 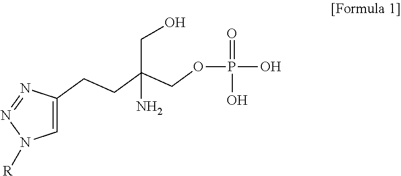
wherein R is C₁₋₁₅ alkyl.

2. The compound of claim 1, wherein the compound represented by Formula 1 is any one selected from the group consisting of the following compounds:
(1) 2-amino-4-(1-hexyl-1H-1,2,3-triazol-4-yl)-2-(hydroxymethyl)butyl dihydrogen phosphate;
(2) 2-amino-2-(hydroxymethyl)-4-(1-octyl-1H-1,2,3-triazol-4-yl)butyl dihydrogen phosphate;
(3) 2-amino-2-(hydroxymethyl)-4-(1-nonyl-1H-1,2,3-triazol-4-yl)butyl dihydrogen phosphate;
(4) 2-amino-4-(1-decyl-1H-1,2,3-triazol-4-yl)-2-(hydroxymethyl)butyl dihydrogen phosphate;
(5) 2-amino-2-(hydroxymethyl)-4-(1-undecyl-1H-1,2,3-triazol-4-yl)butyl dihydrogen phosphate; and
(6) 2-amino-4-(1-dodecyl-1H-1,2,3-triazol-4-yl)-2-(hydroxymethyl)butyl dihydrogen phosphate.

3. The compound of claim 1, wherein the compound represented by Formula 1 is represented by Formula 1a below:

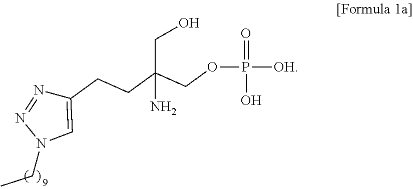 [SMILES: CCn1cc(CCC(N)(CO)COP(=O)(O)O)nn1]

4. An S1PR1 and S1PR4 antagonist represented by Formula 1 below: 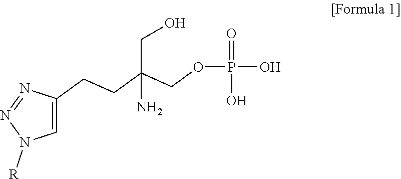
wherein R is C₁₋₁₅ alkyl.

5. The antagonist of claim 4, wherein the compound represented by Formula 1 has at least a 10-fold increase in S1PR1 and S1PR4 binding affinity compared to a compound represented by Formula 2 below: 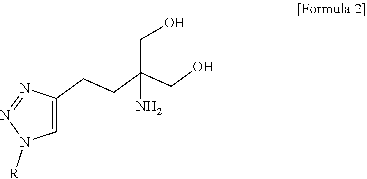
wherein R is C₁₋₁₅ alkyl.

6. The antagonist of claim 4, wherein the compound represented by Formula 1 is represented by Formula 1a below:

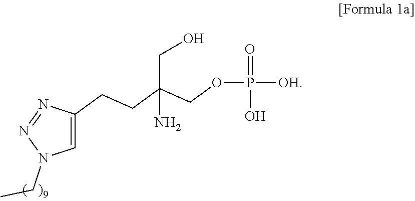

7. A method of preventing or treating inflammatory bowel disease, which comprises administering the compound of claim 1 or a pharmaceutically acceptable salt thereof to a subject in need thereof.

8. The method of claim 7, wherein the inflammatory bowel disease ulcerative colitis (UC) or Crohn's disease (CD).

9. The method of claim 7, wherein the pharmaceutical composition does not cause cardiovascular disease side effects.

10. A method of preventing or treating alopecia areata (AA), which comprises administering the compound of claim 1 or a pharmaceutically acceptable salt thereof to a subject in need thereof.

11. The method of claim 10, wherein the pharmaceutical composition does not cause cardiovascular disease side effects.

12. A method of preventing or treating an autoimmune disease, which comprises administering the compound of claim 1 or a pharmaceutically acceptable salt thereof to a subject in need thereof.

13. The method of claim 12, wherein the autoimmune disease is one or more selected from the group consisting of asthma, type I diabetes, rheumatoid arthritis, polymyalgia rheumatica, ankylosing spondylitis, psoriatic arthritis, multiple sclerosis (MS), interleukin-17 (IL-17) induced dementia, peripheral neuritis, uveitis, autoimmune cytopenia, autoimmune myocarditis, primary cirrhosis, xeroma, fibromyalgia, Goodpasture's syndrome, autoimmune meningitis, Sjogren's syndrome, Addison's disease, alopecia areata (AA), autoimmune hepatitis, autoimmune parotitis, epididymitis, glomerulonephritis, Grave's disease, celiac disease, Guillain-Barr syndrome, Hashimoto's disease, hemolytic anemia, myasthenia gravis, amyotrophic lateral sclerosis, sarcoidosis, spondyloarthropathies, thyroiditis, vasculitis, myxedema, pernicious anemia, antiphospholipid syndrome, post transplantation late and chronic solid organ rejection, and graft-versus-host disease.

14. The method of claim 12, wherein the pharmaceutical composition does not cause cardiovascular disease side effects.

15. A method of preparing the compound represented by Formula 1 below according to claim 1, comprising: 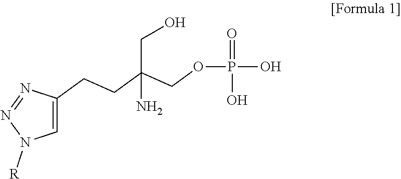
wherein R is C₁₋₁₅ alkyl,
(a) synthesizing a compound represented by Formula 3 below by reacting a compound represented by Formula 2 below with sodium bicarbonate and benzyl chloroformate; 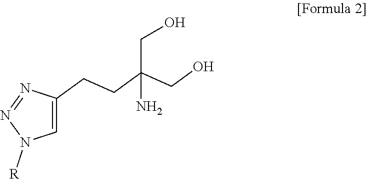
wherein R is C₁₋₁₅ alkyl, 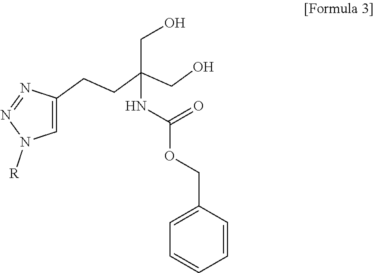
wherein R is C₁₋₁₅ alkyl,
(b) synthesizing a compound represented by Formula 4 below by reacting the compound represented by Formula 3 with tetrabenzyl pyrophosphate; and 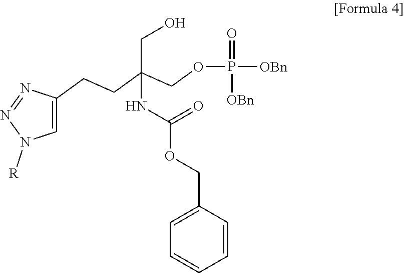
wherein R is C₁₋₁₅ alkyl,
(c) synthesizing the compound represented by Formula 1 by reacting the compound represented by Formula 4 with hydrogen (H₂).

16. The method of claim 15, wherein in (b), the reaction performed by adding N,N-diisopropylethylamine and titanium (IV) butoxide.

17. The method of claim 15, wherein in (c), the reaction performed in the presence of a palladium/carbon catalyst.
